(19) United States
(12) Patent Application Publication
(10) Pub. No.: US 2009/0291149 A1
(43) Pub. Date: Nov. 26, 2009

(54) Method for treatment of overfiltering and leaking blebs with sterile talc

(76) Inventor: Orna Geyer, Tel Aviv (IL)

Correspondence Address: BROWDY AND NEIMARK, P.L.L.C.;624 NINTH STREET, NW, SUITE 300, WASHINGTON, DC, 20001-5303, US

(21) Appl. No.: 12/308,098

(22) PCT Filed: Jun. 7, 2007
(86) PCT No.: PCT/IL2007/000692

§ 371 Date: Dec. 8, 2008

Related U.S. Application Data
(60) Provisional application No. 60/811,795, filed on Jun. 8, 2006.

(51) Int. Cl. A61K 33/12

(52) U.S. Cl. 424/683

(57) ABSTRACT

The present invention provides a method for treatment of overfiltering and leaking blebs in patients after glaucoma surgery comprising administering to said leaking bleb an effective amount of sterile talc, to thereby seal said leaking bleb and prevent bleb leakage.

### FIELD OF INVENTION

The present invention relates to a method for treatment of overfiltering and leading blebs in patients after glaucoma surgery in order to prevent the sight threatening complications thereof.

### BACKGROUND OF THE INVENTION

### Filtering Blebs Following Glaucoma Surgery

Glaucoma leads to blindness by damaging the optic nerve and is considered a leading cause of blindness in the western world. Elevated intraocular pressure is one of the causes of glaucoma and lowering of intraocular pressure is currently the mainstay of glaucoma treatment. This can be achieved with medication and if those fail to control the intraocular pressure, filtration surgery is required.

The most common surgery performed for glaucoma is the trabeculectomy. During this surgery, a hole is made in the scleral wall of the eye to allow fluid to flow out of the eye resulting in lowered intraocular pressure. Scarring of the surgical site can cause operation failure. In order to prevent scarring and increase the success of the surgery, antimetabolites such as mitomycin C (MMC) are used at the time of surgery, inducing the formation of huge cystic filtering blebs. However, after antimetabolites, blebs tend to be large, ischemic with thin walls that are at risk for late bleb rupture causing severe complications such as infections or prolonged hypotony, which carry the most risk for permanent decrease in visual acuity. The incidence of bleb leak is 3.2% per patient-year and the incidence of infection is 1.3% per patient-year. The 5-year probability of developing a bleb is thus 17.9% and of infection is 7.5% (DeBry et al., 2002).

Over the years, many techniques have been attempted to reduce the size of the bleb or to seal the leak in order to prevent the sight threatening complications of the large and leaking blebs, however none of these techniques have proved to be consistently effective. Leen et al. described autologous blood injection into the leaking bleb (Leen et al., 2001); however the treatment was only mildly successful (Burnstein et al., 2001). Grewing and Mester have tried to use fibrin sealant with temporary success (Grewing and Mester, 1997). Geyer described the use of neodymium-YAG laser for treatment of the leaking bleb (Geyer, 1998). Later on, Shoham et al. tried to treat the leakage by placing a contact lens on the leaking bleb, but again with moderate success (Shoham et al., 2000). Amniotic membrane was placed on the bleb by Budenz et al., however it was found to be an uneffective alternative (Budenz et al., 2000). In the absence of a nonsurgical solution to the leaking blebs, many patients undergo a surgical revision and repair in which the bleb is covered by conjunctiva or sclera (Feldman and Altaher, 2004; Harizman et al., 2005). These procedures are considered to be the most reliable and definitive treatment; however leaks can recur or the filtration effect can be lost with subsequent intraocular pressure rise.

### Sterile Talc as a Sclerosing Agent

Sterile talc is a purified naïve hydrated magnesium silicate, produced only for medical purposes. In particular, sterile talc pleurodesis has been employed by chest surgeons for the treatment of pleural effusion (Kolschmann et al., 2005).

According to the literature, talc is the best sclerosing agent available and can be used even after the application of other agents has failed. When injected in a fluid suspension form to the pleural space it causes inflammatory reaction glueing the two pleuras together and leading to obliteration of the space between the layers where the fluid accumulates, thus preventing additional fluid from being accumulated (Kolschmann et al., 2005; Tan et al., 2006).

### SUMMARY OF THE INVENTION

It has now been found, in accordance with the present invention, that sterile talc is useful for treatment of large overfiltering and leaking blebs after glaucoma filtering surgery, thus preventing its complications. It was further found that an intrableb injection of talc suspension or local dispersion of talc on top of the bleb cause irritation which reduces the bleb size. Furthermore, the talc particles temporarily block the holes in the bleb preventing leakage, and the irritant action of the talc causes local inflammation that promotes scarring that permanently seals the leak.

In one aspect, the present invention thus relates to a method for treatment of an overfiltering and leaking bleb in a patient after glaucoma filtering surgery, said method comprising administering to the leaking bleb of the eye of said patient an effective amount of sterile talc, to thereby seal said leaking bleb and prevent bleb leakage.

In another aspect, the present invention relates to use of sterile talc for the preparation of a pharmaceutical composition for administration to the eye to treat an overfiltering and leaking bleb after glaucoma filtering surgery.

In a further aspect, the present invention provides a pharmaceutical composition comprising sterile talc and optionally a pharmaceutically acceptable carrier for treatment of an overfiltering and leaking bleb after glaucoma filtering surgery.

### BRIEF DESCRIPTION OF THE FIGURES

FIG. 1 shows a flat, scarred and vascularized bleb, formed with intraoperative mitomycin-C (0.4 mg/ml) soaked in a cellulose sponge and applied between the sclera and conjunctiva.

FIG. 2 shows an injection of talc slurry into a leaking bleb, using a 25G needle.

FIG. 3 shows the appearance of a treated eye after an intrableb injection of talc suspension. The talc particles inside the bleb are pointed with an arrow, with closure of the leak, and no apparent signs of toxicity are noted.

FIGS. 4A-4C show photomicrographs of hematoxylin-eosin-stained conjuctival epithelium of a normal control eye, X20 (4A) vs. hematoxylin-eosin-stained bleb after peribleb injection of talc slurry (postoperative day 60) (4B-4C). Confluent granuloma under the intact bleb epithelium, composed of giant cells foreign body type engulfing talc particles, are pointed with a black arrow; and the normal third eyelid is pointed with a red arrow, without signs of inflammatory reaction, X20 (4B), X40 (4C) and X200 (inset).

FIGS. 5A-5D show the different stages of a filtration surgery with subconjunctival mitomycin-C (0.4 mg/ml) injection. (5A) Inflated conjunctiva following subconjunctival MMC injection followed by blunt dissection of the subconjunctival space; (5B) A limbal incision 2 mm long was made and the anterior chamber was entered using a Dual Bevel 1.0 mm angled sideport knife (Alcon Laboratories, Inc. Houston, Tex.); (5C) Sectoral iridectomy; and (5D) Bleb at the conclusion of surgery.

FIG. 6 shows improved MMC surgery, wherein the subconjunctival MMC injection at the beginning of surgery resulted in a characteristically avascular and thin bleb that was surrounded by local conjunctival hyperemia.

FIGS. 7A-7B show a leaking bleb. (7A) A hole in the filtering bleb. (7B) Siedel test, using 2% sodium fluorescin, demonstrating the leak. The intact bleb epithelium is stained in green, and the leak site where the fluorescin is washed by aqueous appears blue.

FIG. 8 shows a whisker (pointed with an arrow) inserted at the puncture site in order to maintain the leak. The whisker was passed through the conjunctuval puncture site and a scleral tunnel anteriorly until it was visible in the anterior chamber.

FIG. 9 shows a tube of a 24-gauge intravenous cannula inserted at the puncture site (pointed with an arrow) in order to maintain the leak. A 24G intravenous cannula was passed through the conjunctuval puncture site and a scleral tunnel anteriorly until the cannula needle was visible in the anterior chamber. The cannula needle, was then withdrawn and the extracameral tip was trimmed and bevelled. A 10-0 nylon suture was used to fix the tube to the scleral surface. The conjutiva around the cannula was closed with two interrupted sutures.

FIGS. 10A-10C show an injection of talc slurry via a 24-gauge intravenous cannula, wherein: (10A) A 24G intravenous cannula was introduced through the conjunctiva approximately 2 mm from the filtering bleb and advanced; (10B) the trocar was removed leaving the tube in the bleb; and (10C) the tube was then connected to a 2 ml syringe containing the talc slurry and 0.2 ml of the solution was injected through the tube into the bleb.

### DETAILED DESCRIPTION OF THE INVENTION

In one aspect, the present invention relates to a method for treatment of an overfiltering and leaking bleb in a patient after glaucoma filtering surgery, said method comprising administering to the leaking bleb of the eye of said patient an effective amount of sterile talc, to thereby seal said leading bleb and prevent bleb leakage.

The sterile talc according to the present invention may be any commercially available sterile talc produced for medical purposes, guaranteed asbestos free, free of endotoxins, perfectly controlled granulometry avoiding any migration, non-soluble and either complies with the regulations of the European Pharmacopoeia or approved by the FDA. Examples for such sterile talc are, without being limited to, STERITALC®, manufactured by Novatech SA (France), and Sterile Talc Powder™, manufactured by Bryan Corporation (Woburn, Mass. 01801 U.S.A). Sterile Talc Powder is a sclerosing agent intended for intrapleural administration supplied in a single use 100 mL brown glass bottle, sealed with a gray, 20 mm stopper and covered with a flip-off seal. Each bottle contains a minimum of 5.0 g of Talc USP (Ultra 2000 Talc), either white or off-white to light gray, asbestos-free and brucite-free grade of talc of controlled particle size. The composition of the talc is ≧˜95% talc as hydrated magnesium silicate.

In one embodiment of the present invention, a composition for injection into the leading bleb is provided comprising a suspension of sterile talc in a pharmaceutically acceptable carrier consisting of Sodium Chloride Injection, USP, or any other suitable solution such as balanced salt solution (BSS).

The volume of sterile talc suspension injected into the leaking bleb according to the present invention may be in the range of 0.05 to 0.25 ml, preferably 0.08 to 0.15 ml, more preferably about 0.1 ml, and the amount of sterile talc in the suspension is in the range of 1/30 v/v to ¼ v/v, preferably 1/25 v/v to ⅕ v/v, more preferably 1/10 v/v to ⅕ v/v.

In one preferred embodiment, the intrableb injection is performed through the conjunctiva around the leaking bleb. In particular, the needle may be inserted through the conjunctiva approximately 5 mm from the leaking bleb and advanced until the tip is visible in the bleb, and then slowly injecting the sterile talc suspension into the leaking bleb.

The intrableb injection may be performed using any suitable needle such as, without being limited to, a 25-30G needle, preferably a 25-27G needle.

In a more preferred embodiment, the talc suspension is delivered into the leading bleb through a tube of an intravenous cannula. In particular, an intravenous cannula may be introduced through the conjunctiva approximately 2 mm from the leading bleb and advanced until the tip is visible in the bleb, and then slowly injecting the sterile talc suspension, contained in a syringe connected to the tube of said intravenous cannula, into the bleb.

The intravenous cannula used according to the present invention may be any suitable intravenous cannula such as, without being limited to, a 24-26G intravenous cannula, preferably a 24G intravenous cannula.

It should be noted that in some cases, a small leakage may still be observed following a single injection. In these cases, an additional intrableb talc injection(s) may be required.

In certain cases, in order to simplify the treatment procedure and to avoid the pain associated with an intrableb injection, the administration of sterile talc to the leaking bleb may be performed by local dispersion of the talc on top of the leaking bleb.

Thus, in another embodiment, the sterile talc according to the present invention is locally dispersed on top of the leaking bleb.

In such cases, at least part of the sterile talc dispersed is expected to enter the leaking bleb, causing irritation, which will reduce the bleb size and temporarily block the holes in the bleb preventing leakage, while most of the sterile talc dispersed is washed with the tears. Thus, in many cases when sterile talc dispersing is utilized, the treatment procedure would have to be repeated several times until the required result is achieved.

In another aspect, the present invention relates to use of sterile talc for the preparation of a pharmaceutical composition for administration to the eye to treat an overfiltering and leaking bleb after glaucoma filtering surgery.

In a further aspect, the present invention provides a pharmaceutical composition comprising sterile talc and optionally a pharmaceutically acceptable carrier for treatment of an overfiltering and leaking bleb after glaucoma filtering surgery.

In preferred embodiments, the pharmaceutical compositions of the present invention comprise a sterile talc suspension wherein the sterile talc is suspended in a suitable solution such as Sodium Chloride Injection, USP, or preferably, balanced salt solution (BSS).

The invention will now be illustrated by the following non-limiting Examples.

### EXAMPLES

### Material and Methods

(i) Animal Care.

New Zealand white rabbits, weighing between 2 to 4 kg, were housed in separate clear cages (one rabbit per cage) that allowed them to see and hear each other. The cages had plastic floor and a shelter where the rabbits could hide, and contained white pine shavings for bedding. The cages were covered loosely with air filters and the environment was kept at 21° C. with 12 hours light and 12 hours dark cycle. All rabbits were fed with a standard food mixture.

(ii) Anesthesia Protocol.

General anesthesia was induced with an intramuscular injection of ketamine (35 mg/kg), xylazine (5 mg/kg), and acepromazine (1 mg/kg). Analgesia was induced with a subcutaneous injection of butorphanol (0.1-0.5 mg/kg) to treat pre and post operative pain.

### Example 1

### The Effect of Intrableb Injection of Talc on Leaking Blebs in Rabbits—Pilot Study

Four New Zealand white rabbits were anesthesized as described in Materials and Methods, and trabeculectomy operation with mitomycin C (MMC) was performed as follows.

A standard posterior lip sclerectomy was performed on 4 left eyes of 4 different rabbits, and a superior limbal-based conjunctival flap was raised. Intraoperative MMC (0.4 mg/ml) soaked in a cellulose sponge was applied between the sclera and conjunctiva for 5 minutes, and the treated area was then thoroughly irrigated with 30 ml of balanced salt solution (BSS). The concentration of MMC was chosen in view of a previous publication by Khaw et al., 1993, in which the use of this MMC dose in the rabbit model produced long-lasting blebs and reduction of intraocular pressure.

A limbal, 2-mm incision was made and the anterior chamber was penetrated. A 1.5-mm Kelly's Descemet membrane punch was used to remove a standard posterior block of tissue. A peripheral iridectomy was made and the conjunctival incision was then closed with a running 8-0 coated Vicryl polyglactin suture on a BV130-4 needle (Ethicon, Somerville, N.J.). A 30-gauge paracentesis of the anterior chamber was made and balanced salt solution (BSS) was injected into the anterior chamber in order to confirm the presence of a patent scierectomy and to inflate the bleb. The conjunctival incision and the bleb were inspected to rule out any leaks, and Maxitrol® ointment was applied at the end of the surgery, and during the following week.

Post operative examinations were performed under anesthesia. The size of the bleb was graded as not present (0); low (1), if there was minimal elevation but no cystic changes; moderate (2); or high (3), depending on the degree of elevation, avascularity and cystic changes. The anterior chamber depth was estimated, Fluorescein was used to determine the presence of any bleb leaks.

Seven days post operation all the eyes were examined under anesthesia as described above. In all cases, the blebs were flat vascular and showed evidence of scarring and failure (graded as 0). Surgical revision was then performed under anesthesia, during which 0.1 ml MMC (0.4 mg/ml) was injected into the bleb.

Ten days post operation, moderate blebs (grade 2) were observed in all 4 eyes. The intraocular pressure was low on palpation and the anterior chamber was shallow, having a mean depth of approximately 50% compared with the fellow eye in all treated animals.

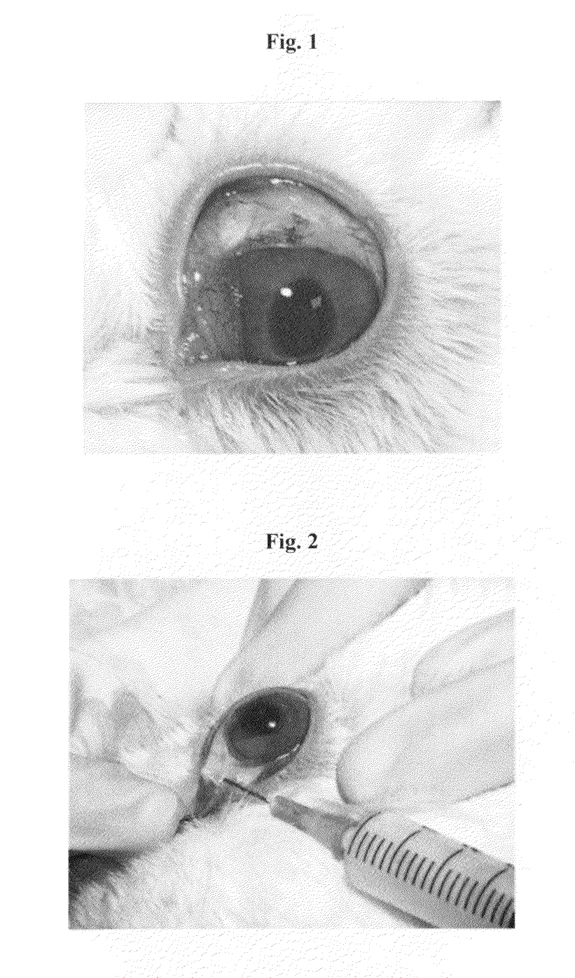

At that time, blebs were punctured with a Beaver blade (Becton, Dickinson and Co., NJ, USA), to yield a 1 mm incision, and using the Seidel test, in which 2% sodium fluorescein is used in order to check for leaks such as post-trabeculectomy, all were found to be leaking (Seidel-positive). Examination of the blebs 24 hours later revealed no leak in all cases, and the blebs were repeatedly punctured in the same spot. In order to establish a stable leak that does not self-seal, multiple (2-4) bleb punctures had to be made over the next days in all cases. A leaking bleb was considered only when a stable leak was observed over 2 weeks. At this time, the bleb was flat and the anterior chamber depth varied from less than half deep to fully deep, with a mean depth of approximately 50% compared with the fellow eye. FIG. 1 shows a flat, scarred and vascularized bleb, formed with intraoperative MMC (0.4 mg/ml) soaked in a cellulose sponge and applied between the sclera and conjunctiva.

After confirming leaking blebs, the animals were anesthesized and the treated eyes received intrableb talc injection, using a 25-gauge needle. The needle was inserted through the conjunctiva approximately 2 mm from the filtering bleb and advanced until the tip was visible in the bleb. In each case, 0.2 ml of sterile talc suspension containing ⅕ v/v sterile talc in physiological saline was injected (FIG. 2). The rationale for this concentration was to achieve good talc dissolution.

The eyes were reexamined an hour later. Maxitrol® ointment was applied after all injections, as well as during the following week. The treated eyes were examined daily for one week, and all were found to be leaking. Repeated intrableb talc injections were administered as described above, and examinations were performed on a weekly basis. In cases the leak persisted during the follow-up examination, a repeat injection was performed (one week apart) until the leak was sealed. An average of 4 injections were performed until all bleb leaks were sealed.

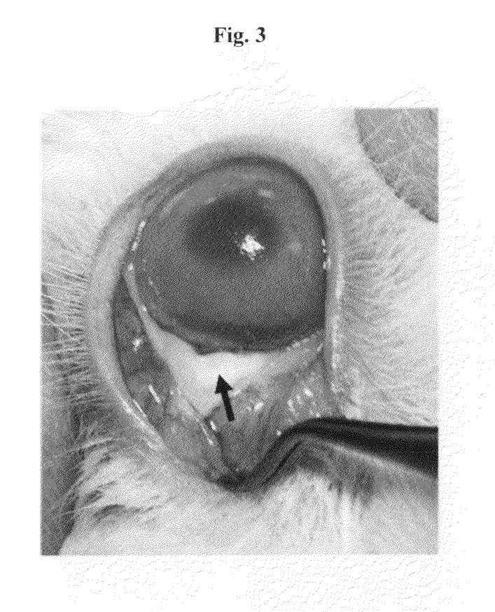

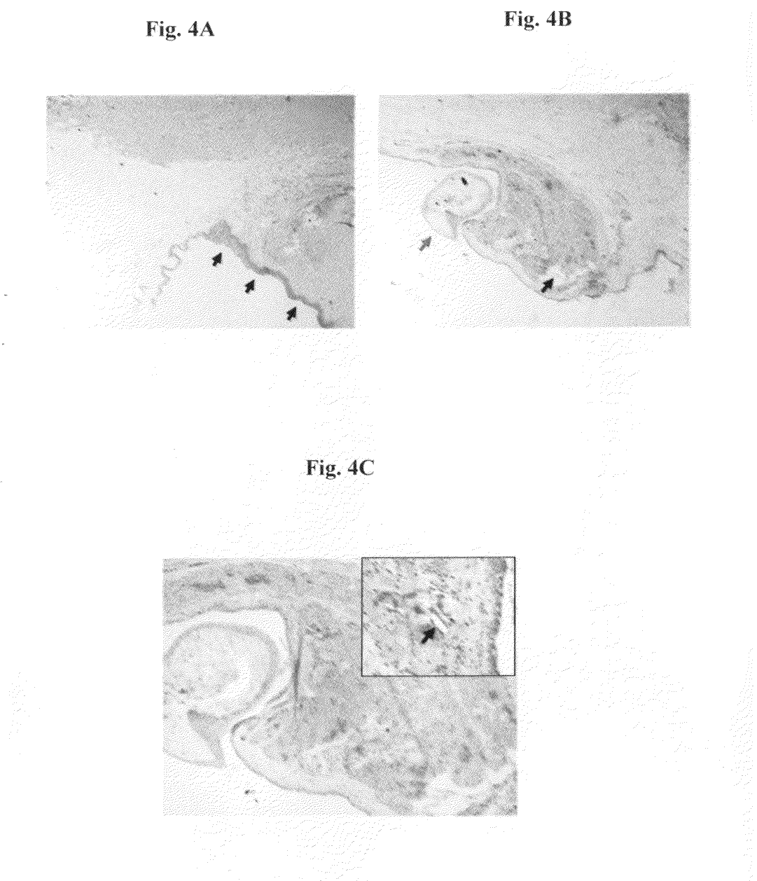

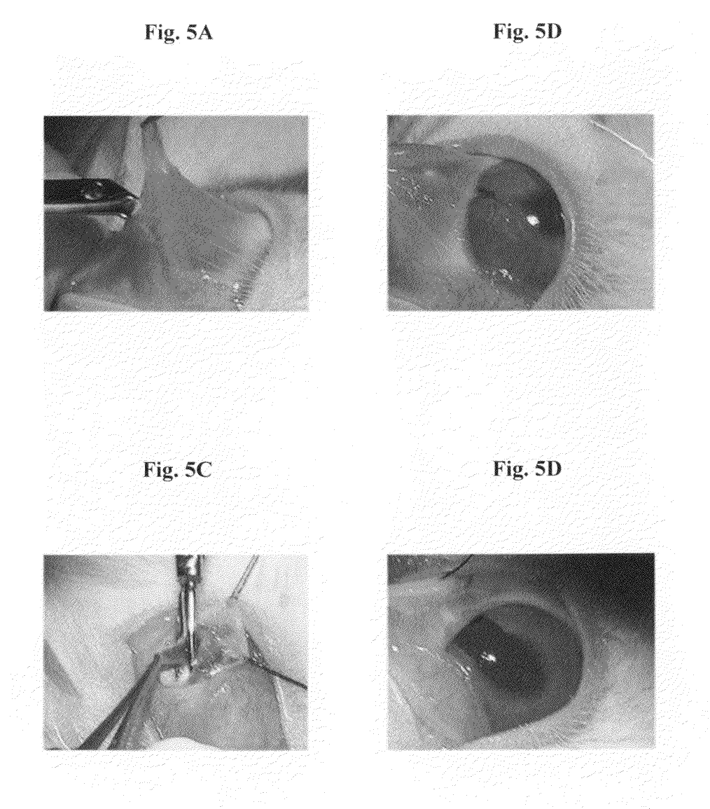

The blebs were followed for an additional month to ensure that the leaks did not recur. At that time, the treated eyes were soft on palpation; the blebs appeared localized avascular with white precipitates and moderately elevated (graded as 1); all the anterior chambers were fully formed; and no apparent signs of toxicity were noted (FIG. 3).

The rabbits were then sacrificed and the left eye of each was removed for histologic study. Histologic examination revealed talc granuloma at the site of the bleb. FIGS. 4B-4C show photomicrographs of a hematoxylin-eosin-stained bleb on postoperative day 60, demonstrating the confluent granuloma under the intact bleb epithelium, composed of giant cells foreign body type engulfing talc particles, and the normal third eyelid, without signs of inflammatory reaction.

### Example 2

### The Effect of Intrableb Injection of Talc on Leaking Blebs in Rabbits—Improved Model

Six New Zealand white rabbits were anesthesized as described in Materials and Methods, and trabeculectomy operation with MMC was performed as described in Example 1 above, with the difference that in the present experiment, MMC was injected subconjunctivally at the site of the planned surgery at the beginning of the operation instead of intraoperatively applying the drug with a sponge, and the conjunctival was placed over the incision site and no suture was used in order to close the conjunctiva. Maxitrol® ointment was applied at the end of the surgery, and during the following week (FIGS. 5A-5D).

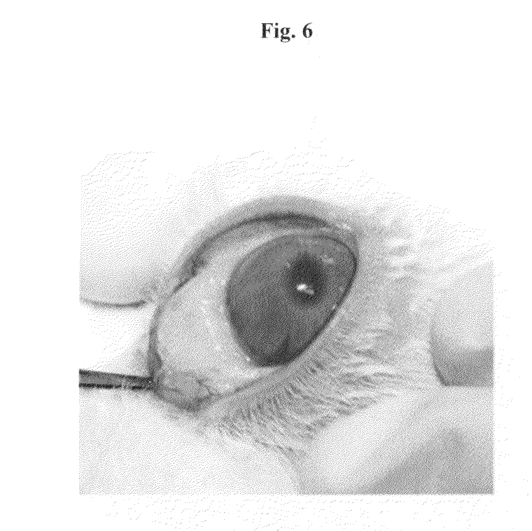

Seven days post operation all the eyes were examined as described in Example 1 above. In all cases, the blebs were diffuse, elevated, and avascular (graded as 3), as shown in FIG. 6.

Ten days post operation, moderate blebs (grade 2) were observed in all 6 eyes. The intraocular pressure was low on palpation and anterior chamber was shallow, having a mean depth of approximately 50% compared with the fellow eye in all treated animals. Eyes were followed for additional 7 days to ensure that the blebs were functioning and that no leaks were present. Fourteen days post operation, no changes were observed in the blebs, the intraocular pressure in all the treated eyes was low on palpation and the anterior chambers were shallow, with a mean depth of approximately 50% compared with the fellow eye.

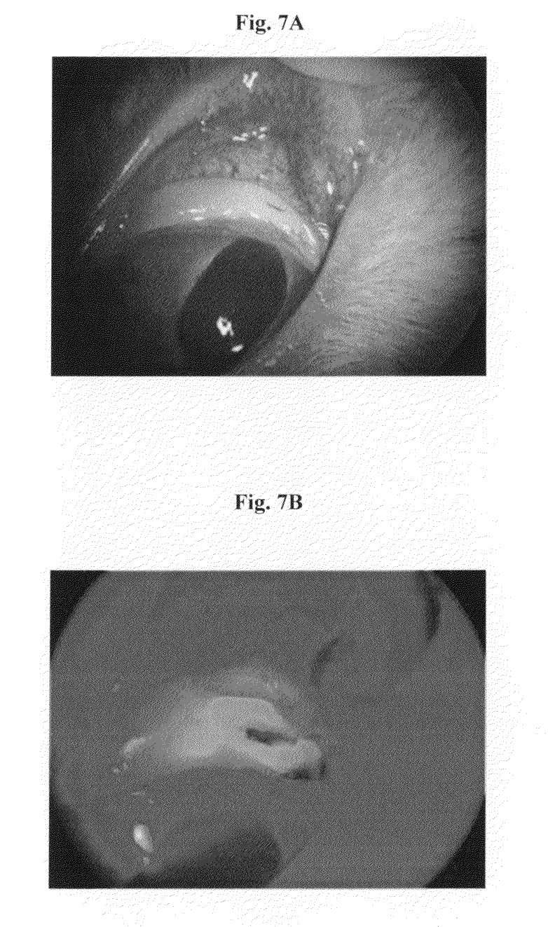

At that time, blebs were punctured with a Beaver blade (Becton, Dickinson and Co., NJ, USA), to yield a 2 mm incision. All animals were examined one hour following the puncture, and an examination 24 hours after puncture revealed leaking blebs in all the treated eyes, as shown in FIGS. 7A-7B. The blebs were followed for an additional week to ensure that the leak did not heal. At this time, all blebs were smaller and appeared cystic, localized and avascular. The eyes were soft on palpation, and the anterior chambers depth varied from less than half deep to fully deep, with a mean depth of approximately 50% compared with the fellow eye.

Talc slurry was prepared as described in Example 1 above and injected into the leaking blebs, using a 25-gauge needle, as described above. The eyes were reexamined an hour later, and Maxitrol® ointment was applied after all injections, as well as during the following week.

The treated eyes were examined daily for a week, and then weekly, until no leak was observed. In case the leak persisted, additional intrablec talc injections were performed on a weekly basis, as described above, until the leak was sealed. In particular, 1 eye received a single injection, 3 eyes received 2 injections and 3 eyes received 3 injections. Bleb leaks were sealed by days 15-20 following the first injection. At that time, the treated eyes were soft on palpation; the blebs appeared localized avascular with white precipitates and moderately elevated (graded as 1); all the anterior chambers were fully formed; and no apparent signs of toxicity were noted.

The rabbits were then sacrificed and the left eye of each was removed for histologic study.

### Example 3

### The Effect of Intrableb Injection of Talc on Leaking Blebs in Rabbits—Extended Study

Twelve New Zealand white rabbits were anesthesized as described in Materials and Methods, and trabeculectomy operation with MMC was performed according to the revised surgical protocol established and described in Example 2 above. Fourteen days post operation, large, functioning filtering blebs with no leaks were observed in all the treated animals.

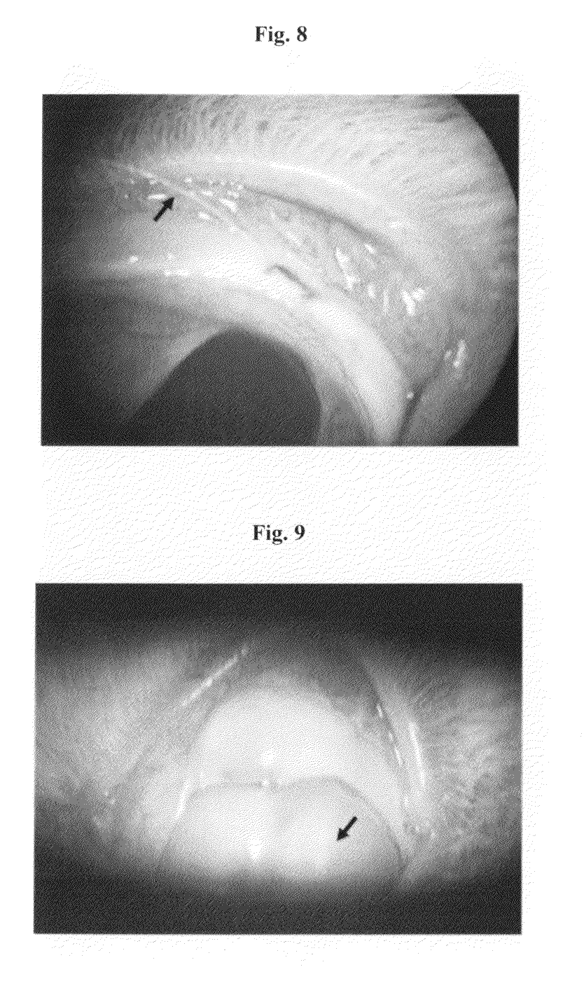

At that time, blebs were punctured as described in Example 2 above to yield a 2 mm incision; however, the following day, no leak was observed in all treated eyes. Then, all the blebs were punctured once again and a rabbit whisker (FIG. 8) or a tube of a 24-gauge intravenous cannula (FIG. 9) was introduced at the puncture site in order to maintain the leak. The tube was secured to the sclera using an encircling 10-0 nylon suture (Ethicon Inc., Somerville, N.J.). Two weeks later, leak was observed in 8 out of the 12 treated eyes.

The eyes with the leaking bleb were randomly assigned to receive intrableb injection of either talc slurry (⅕ v/v sterile talc in physiological saline) or physiological saline; however, the mode of injection was changed compared with the previous experiments. In particular, in the pilot and the second experiment described in Examples 1-2 above, the talc suspension was delivered to the leaking blebs with a 25G needle; however, multiple injections were needed in order to seal the blebs. In view of this, and since talc powder was found in both the syringe and the needle following these injections, it is possible that the small caliber of the needle allowed mainly the passage of the watery content of the slurry with very little talc, and that only a low amount of talc was, in fact, delivered into the blebs.

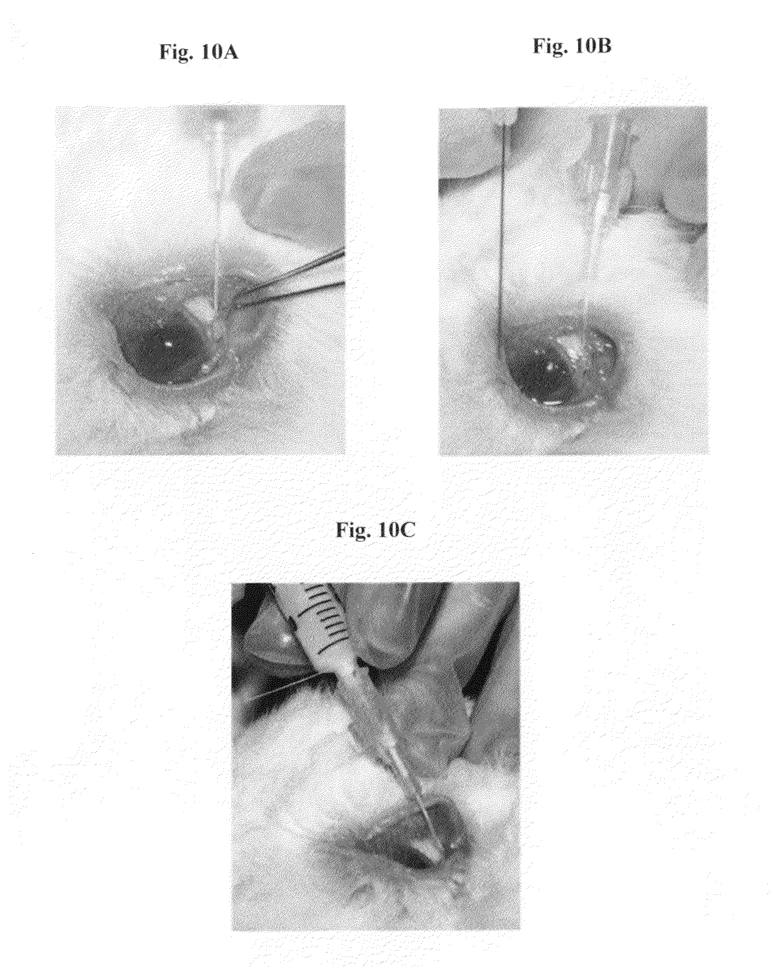

Contrary to the method used in the first two experiments, in this experiment the talc slurry was delivered through a tube of a 24-gauge intravenous cannula, as shown in FIGS. 10A-10C. In particular, a 24-gauge intravenous cannula (Becton Dickinson Vascular Access Inc., UT, USA) was introduced through the conjunctiva approximately 2 mm from the filtering bleb and advanced until the tip was visible in the bleb and the trocar was removed. The tube was then connected to a syringe containing the suspension for injection, and 0.2 ml of the solution was injected through the tube into the bleb. Following each one of the injections, no talc remnants were seen neither in the syringe nor in the tube.

The eyes were reexamined one hour late. Maxitrol® ointment was applied after all injections, and during the following week.

One week following the intrableb injections, all blebs into which saline was injected were leaking. In two of the four blebs into which the talc slurry was injected, the leak was closed. The eyes were soft on palpation; the blebs appeared localized avascular with white precipitates and moderately elevated (graded as 1); all the anterior chambers were fully formed, and no apparent signs of toxicity were noted. The other two blebs treated with talc slurry were still leaking, but the leak was much smaller. In these cases, additional intrablec talc injections were performed, as described above.

### Example 4

### Treatment Protocol of Leaking Blebs in Humans, Using Intrableb Injection of Talc

The intrableb injection of the sterile talc suspension is performed in a treatment room under an operating microscope as follows.

Sterile talc suspension is prepared by suspending sterile talc in balanced salt solution (BSS) in the range of 1/25 v/v to ⅕ v/v in a 2 ml syringe.

The treated eye is anesthetized with oxybuprocaine hydrochloride 0.4% eye drops (3 doses, 5 minutes apart, beginning 15 minutes before injection). One minute prior to the injection, 0.25 inch of lidocaine 2% gel is applied in the conjunctival fornix, and then, 2 drops of topical ofloxacin hydrochloride 0.3% and povidone iodine 5% are applied. A sterile lid speculum is placed in the eye.

A 24-gauge to 26-gauge intravenous cannula (Abbocath, Sligo, Republic of Ireland) is introduced through the conjunctiva approximately 2 mm from the filtering bleb and advanced until the tip is visible in the bleb. The trocar is removed, and the tube is then connected to the syringe containing the suspension for injection. A volume of 0.05 to 0.25 ml, preferably 0.1 ml of the solution is injected through the tube into the bleb.

Following the injection of the sterile talc suspension, an antibiotic ointment and an ocular patch are applied, and the eye is reexamined 1 hour later. Topical antibiotic is applied for the following week.

### REFERENCES

Budenz, D. L. Barton, K. Tseng, S. C., Amniotic membrane transplantation for repair of leaking glaucoma filtering blebs, Am J. Ophtalml., 2000, 130(5), 580-588
Burnstein, A. WuDunn, D. Ishii, Y. Jonescu-Cuypers, C. Cantor, L. B., Autologous blood injection for late-onset filtering bleb leak, Am J. Opthalmol., 2001, 132(1), 36-40
DeBry, P. W. Perlins, T. W. Heatley, G. Kaufman, P. Brumback, L. C., Incidence of late-onset bleb-related complications following trabeculectomy with mitomyxin, Arch Opthalmol., 2002, 120(3), 297-300
Feldman, R. M. Altaher, G., Management of late-onset bleb leaks, Curr Opin Ophtalmol., 2004, 15(2), 151-154
Geyer, O., Management of large, leaking, and inadvertent filtering blebs with the neodymium: YAG laser, Ophtalmology, 1998, 105(6), 983-987
Grewing, R. Mester, U., Fibrin sealant in the management of complicated hypotony after trabeculectomy, Ophthalmic surg lasers., 1997, 28(2), 124-127
Harizman, N. Ben-Cnaan, R. Goldenfeld, M. Levkovitch-Verbin, H. Melamed, S., Donor scleral patch for treating hypotony due to leading and/or overfiltering blebs, J Glaucoma, 2005, 14(6), 492-496
Khaw, P. T. Doyle, J. W. Sherwood, M. B. Smith, M. F. McGorray, S., Effects of intraoperative 5-fluorouracil or mitomycin-C on glaucoma filtration surgery in the rabbit, Opthalmology, 1993, 100(3), 367-372
Kolschmann, S. Ballin, A. Gillissen, A., Clinical efficacy and safety of thoracoscopic talc pleurodesis in malignant pleural effusions, Chest, 2005, 128(3), 1431-1435
Leen, M. M. Moster, M. R. Kats, L. J. Terebuh, A. K. Schmidt, C. M. Spaeth, G. L., Management of overfiltering and leaking blebs with autologous blood injection, Arch Ophthalmol., 1995, 113(8), 1050-1055
Shoham, A. Tessler, Z. Finkelman, Y. Lifshitz, T., Large soft contact lenses in the management of leading blebs, CLAO J., 2000, 26(1), 37-39
Tan, C. Sedrakyan, A. Browne, J. Swift, S. Treasure, T., The evidence on the effectiveness of management for malignant pleural effusion: a systematic review, Eur J Cardiothorac Surg., 2006, 29(5), 829-838

1. A method for treatment of an overfiltering and leaking bleb in a patient after glaucoma filtering surgery, said method comprising administering to the leaking bleb of the eye of said patient an effective amount of sterile talc, to thereby seal said leaking bleb and prevent bleb leakage.

2. The method of claim 1, wherein said sterile talc is in the form of a suspension for injection into the leaking bleb.

3. The method of claim 2, wherein said sterile talc is suspended in balanced salt solution (BSS).

4. The method of claim 2, wherein the suspension is injected through the conjunctiva around the leaking bleb.

5. The method of claim 4, wherein the volume of said suspension is in the range of 0.05 to 0.25 ml and the amount of sterile talc in said suspension is in the range of 1/30 v/v to ¼ v/v.

6. The method of claim 5, wherein the volume of said suspension is in the range of 0.08 to 0.15 ml and the amount of sterile talc in said suspension is in the range of 1/25 v/v to ⅕ v/v.

7. The method of claim 6, wherein the volume of said suspension about 0.1 ml and the amount of sterile talc in said suspension is in the range of 1/10 v/v to ⅕ v/v.

8. The method of claim 2, wherein said suspension is injected through a tube of a 24-26G intravenous cannula.

9. The method of claim 8, wherein said suspension is injected through a tube of a 24G intravenous cannula.

10. The method of claim 2, wherein said suspension is injected with a 25-30G needle.

11. The method of claim 10, wherein said suspension is injected with a 25-27G needle.

12. The method of claim 1, wherein said sterile talc is locally dispersed on top of the leaking bleb.

13-23. (canceled)

24. A pharmaceutical composition comprising sterile talc suspended in balanced salt solution.

25-26. (canceled)

27. The pharmaceutical composition of claim 24, for injection through the conjunctiva around an overfiltering and leaking bleb after glaucoma filtering surgery.

28. The pharmaceutical composition of claim 24, wherein the amount of sterile talc in said suspension is in the range of 1/30 v/v to ¼ v/v.

29. The pharmaceutical composition of claim 28, wherein the amount of sterile talc in said suspension is in the range of 1/25 v/v to ⅕ v/v.

30. The pharmaceutical composition of claim 29, wherein the amount of sterile talc in said suspension is in the range of 1/10 v/v to ⅕ v/v.

31-34. (canceled)
